(19) United States
(12) Patent Application Publication
(10) Pub. No.: US 2012/0065707 A1
(43) Pub. Date: Mar. 15, 2012

(54) NERVE STIMULATION APPARATUS

(75) Inventors: Ryuichi YORIMOTO, Tokyo (JP); Hiroki HIBINO, Tokyo (JP)

(73) Assignee: OLYMPUS CORPORATION, Tokyo (JP)

(21) Appl. No.: 13/034,751

(22) Filed: Feb. 25, 2011

(30) Foreign Application Priority Data
Sep. 9, 2010 (JP) 2010-202180

(51) Int. Cl. A61N 1/36

(52) U.S. Cl. 607/62

(57) ABSTRACT

A cardiac activity state is correctly determined and the vagus nerve is stimulated at an appropriate timing. Provided is a nerve stimulation apparatus including a detector that detects an electrocardiac signal; a nerve stimulation unit that generates an electrical pulse for stimulating the vagus nerve; and a controller that determines a cardiac activity state on the basis of the electrocardiac signal detected by the detector and controls the nerve stimulation unit on the basis of the determination result; wherein the controller has a determination period for determining the cardiac activity state and a non-determination period for not determining the cardiac activity state, and wherein the controller allows the electrical pulse to be generated from the nerve stimulation unit while controlling a generation period of the electrical pulse so as to include it within the non-determination period.

### BACKGROUND OF THE INVENTION

1. Field of the Invention

The present invention relates to a nerve stimulation apparatus.

This application is based on Japanese Patent Application No. 2010-202180, the content of which is incorporated herein by reference.

2. Description of Related Art

Conventional heart treatment devices that prevent tachyarrhythmia and regulate the heart rate are known (for example, see Japanese Unexamined Patent Application, Publication No. 2004-173790). In general, cardiac activity is increased when signal activity of the sympathetic nerve is increased, and cardiac activity is decreased when signal activity of the vagus nerve is increased. Japanese Unexamined Patent Application, Publication No. 2004-173790 discloses a heart treatment device that stimulates the heart with an electrical pulse when the heart rate is lower than a predetermined rate, and when the heart rate is higher than a predetermined rate, stimulates the vagus nerve with an electrical pulse without stimulating the heart. By doing so, the heart rate is regulated at a lower value.

### BRIEF SUMMARY OF THE INVENTION

An object of the present invention is to provide a nerve stimulation apparatus that is capable of determining a cardiac activity state correctly and that is capable of stimulating the vagus nerve at an appropriate timing.

The present invention employs the following solutions.

One aspect of the present invention is a nerve stimulation apparatus including a detector that detects an electrocardiac signal; a nerve stimulation unit that generates an electrical pulse for stimulating the vagus nerve; and a controller that determines a cardiac activity state on the basis of the electrocardiac signal detected by the detector and controls the nerve stimulation unit on the basis of the determination result; wherein the controller has a determination period for determining the cardiac activity state and a non-determination period for not determining the cardiac activity state, and wherein the controller allows the electrical pulse to be generated from the nerve stimulation unit while controlling a generation period of the electrical pulse so as to be included within the non-determination period.

In the above-mentioned aspect, the non-determination period may be a period from the point at which the electrocardiac signal containing the R wave is detected with the detector to the point at which the electrocardiac signal containing the T wave is detected with the detector

In the above-mentioned aspect, the controller may extend the non-determination period according to the determination result of the cardiac activity state.

In the above-mentioned aspect, the controller may vary the non-determination period according to a generation condition of the electrical pulse.

According to the present invention, an advantage is afforded in that it is possible to correctly determine the cardiac activity state and to stimulate the vagus nerve at an appropriate timing.

### BRIEF DESCRIPTION OF THE SEVERAL VIEWS OF THE DRAWINGS

FIG. 1 is a diagram showing the overall configuration of a nerve stimulation apparatus according to an embodiment of the present invention.

FIG. 2 is a block diagram of the apparatus main body shown in FIG. 1.

FIG. 3 is a diagram showing a state where an electrical pulse is generated during a generation period that is shorter than a non-determination period.

FIG. 4 is a diagram showing a state where an electrical pulse is generated during a generation period having substantially the same duration as a non-determination period.

FIG. 5 is a flowchart for stimulating the vagus nerve with a nerve stimulation apparatus according to an embodiment of the present invention.

FIG. 6 is a diagram showing, as a reference example, a state where an electrical pulse is generated during a generation period that is longer than a non-determination period.

### DETAILED DESCRIPTION OF THE INVENTION

A nerve stimulation apparatus according to the present invention is provided with a detector that detects an electrocardiac signal, a nerve stimulation unit that generates an electrical pulse for stimulating the vagus nerve, and a controller that determines a cardiac activity state on the basis of the electrocardiac signal detected by the detector and controls the nerve stimulation unit on the basis of the determination result; the controller has a determination period for determining the cardiac activity state and a non-determination period for not determining the cardiac activity state, and the controller allows the electrical pulse to be generated from the nerve stimulation unit while controlling a generation period of the electrical pulse so as to include it within the non-determination period.

By this configuration, with the operation of the controller, the vagus nerve is stimulated by the electrical pulse generated from the nerve stimulation unit according to the cardiac activity state that has been determined on the basis of the electrocardiac signal. By controlling the generation period of the electrical pulse so as to be included within the non-determination period, the generation of the electrical pulse is stopped during the determination period, and so it is possible to prevent interference of the electrical pulse with the detector via the living body. Thereby, it is possible to avoid inclusion of the electrical pulse with the electrocardiac signal that is detected with the detector during the determination period.

Therefore, in the controller, it is possible to correctly determine the cardiac activity state on the basis of the correct electrocardiac signal that has been detected by the detector without being influenced by the electrical pulse, and therefore, it is possible to stimulate the vagus nerve at an appropriate timing.

In the above-mentioned nerve stimulation apparatus, the non-determination period may be a period from the point at which the electrocardiac signal containing the R wave is detected by the detector to the point at which the electrocardiac signal containing the T wave is detected by the detector.

The R wave appears when the ventricles contract and the T wave appears when the contracted heart relaxes. Therefore, by setting a period from the point at which the electrocardiac signal containing the R wave is detected by the detector to the point at which the electrocardiac signal containing the T wave is detected by the detector as the non-determination period, it is possible to prevent the controller from undesirably determining the electric potential variations due to pacing, spontaneous cardiac activity, or the like.

In the above-mentioned nerve stimulation apparatus, the controller may extend the non-determination period in accordance with the determination result of the cardiac activity state.

With this configuration, when it is necessary to generate the electrical pulse outside the preset non-determination period as a result of the determination of the cardiac activity state with the controller, by extending the non-determination period, it is possible to allow the generation period of the electrical pulse to be included within the extended non-determination period. By doing so, it is possible to stimulate the vagus nerve correctly and sufficiently while preventing the influence of interference of the electrical pulse, without reducing the necessary generation period of the electrical pulse.

In the above-mentioned nerve stimulation apparatus, the controller may vary the non-determination period according to the generation conditions of the electrical pulse.

The generation conditions of the electrical pulse vary depending on the treatment of the patient with cardiac disease, and the influence of the interference of the electrical pulse with to the detector also vary depending on the treatment status. Therefore, by varying the non-determination period according to the generation conditions of the electrical pulse with the controller, it is possible to perform nerve stimulation appropriate for the treatment status. The generation conditions of the electrical pulse include the position of the vagus nerve to be stimulated, the timing for generating the electrical pulse, and so forth.

A nerve stimulation apparatus according to an embodiment of the present invention will be described below, with reference to the drawings.

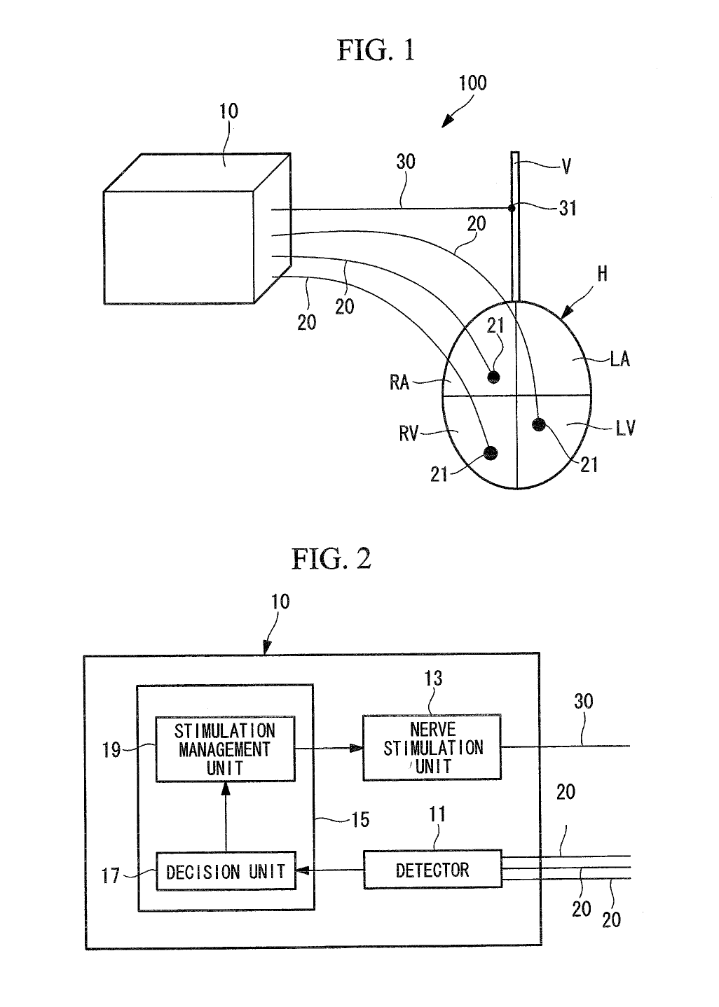

As shown in FIG. 1, a nerve stimulation apparatus 100 according to this embodiment is provided with an apparatus main body 10, three cardiac leads 20 that are extended from the apparatus main body 10 to electrically connect the apparatus main body 10 and the heart H, and a nerve stimulation lead 30 that electrically connects the apparatus main body 10 and the vagus nerve V.

The respective cardiac leads 20 are electrically connected to respective electrodes 21 that are formed of a conductive material and attached to RA (the right atrium), RV (the right ventricle), and LV (the left ventricle). The nerve stimulation lead 30 is electrically connected to an electrode 31 that is formed of a conductive material and attached to the vagus nerve V. The reference symbol LA indicates the left atrium.

As shown in FIG. 2, the apparatus main body 10 is provided with a detector 11 that detects an electrocardiac signal, a nerve stimulation unit 13 that generates an electrical pulse for stimulating the vagus nerve V, and a controller 15 that controls the nerve stimulation unit 13 on the basis of the electrocardiac signal detected by the detector 11.

The three cardiac leads 20 are electrically connected to the detector 11. The detector 11 continuously detects the electrocardiac signal sent from the respective electrodes 21 through the respective cardiac leads 20 and outputs the timing information of the beating of the heart H that is detected from the electrocardiac signal to the controller 15.

The nerve stimulation lead 30 is electrically connected to the nerve stimulation unit 13. The nerve stimulation unit 13 supplies the electrical pulse having a preset voltage value and pulse duration to the electrode 31 via the nerve stimulation lead 30 in accordance with an instruction from the controller 15.

The controller 15 is provided with a decision unit 17 that determines the activity state of the heart H on the basis of the timing information of the beating of the heart H that is input from the detector 11 and a stimulation management unit 19 that manages the generation of the electrical pulse by the nerve stimulation unit 13.

The decision unit 17 sets a determination period for determining the activity state of the heart H and the non-determination period for not determining the activity state of the heart H. It is preferable that the determination period and the non-determination period be set such that, by taking, for example, the R wave and the T wave that appear in the electrocardiac signal into consideration, electric potential variations due to pacing, spontaneous cardiac activity, or the like will not be undesirably determined.

In addition, the decision unit 17 determines whether the heart H is in the normal state or in a tachycardiac state based on the electrocardiac signal detected by the detector 11 during the determination period. More specifically, the decision unit 17 has a reference rate for setting a predetermined heart rate for every patient with cardiac disease. The decision unit 17 determines whether the heart rate is higher than the reference rate based on the timing information of the beating of the heart H that is sent from the detector 11. For example, the decision unit 17 determines that the heart H is in the tachycardiac state if the heart rate is higher than the reference rate.

When the decision unit 17 determines that the heart H is in the tachycardiac state, the stimulation management unit 19 outputs a generation signal of the electrical pulse to the nerve stimulation unit 13 in accordance with its determination result, in other words, in accordance with the activity state of the heart H, and allows the electrical pulse to be generated during the non-determination period.

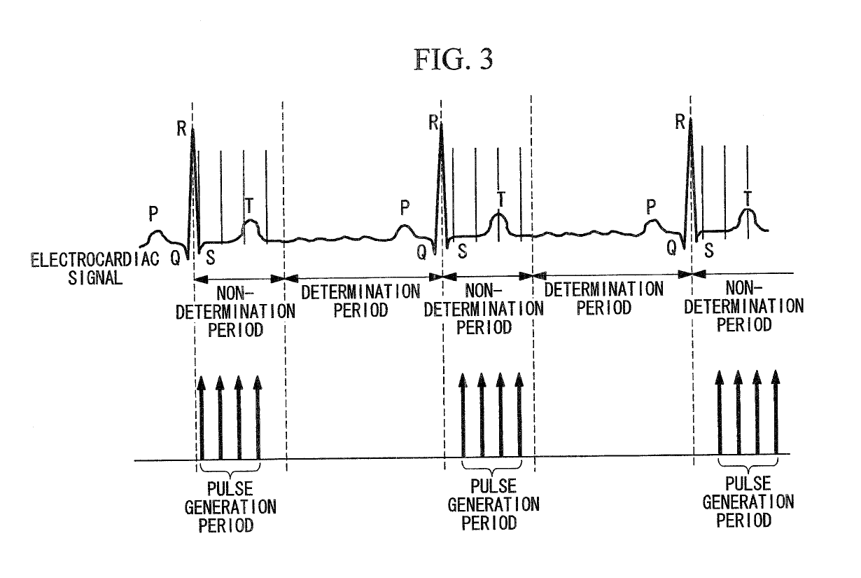

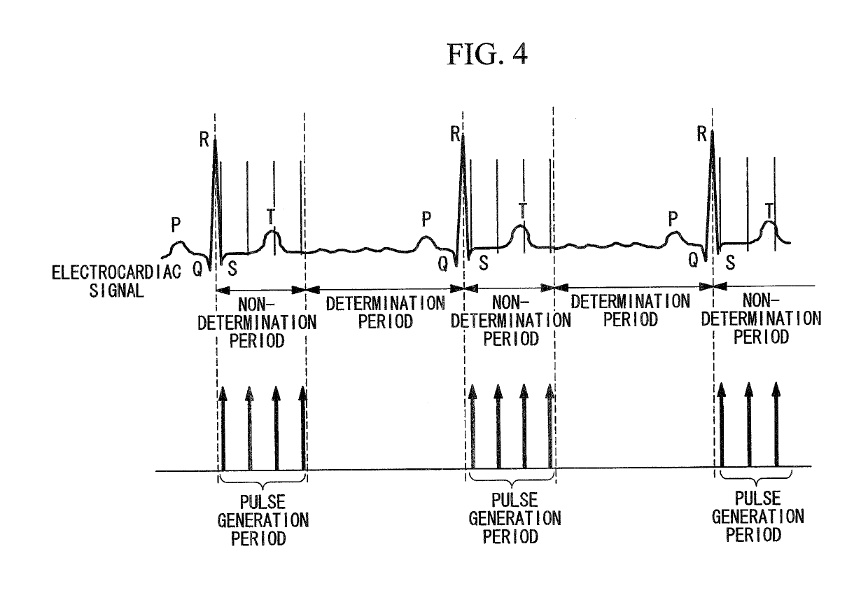

In addition, the stimulation management unit 19 controls the generation period of the electrical pulse so as to be included within the non-determination period by the decision unit 17 and so as not to be included within the determination period. For example, as shown in FIG. 3, the stimulation management unit 19 sets the duration of the generation period of the electrical pulse to be shorter than that of the non-determination period, or, as shown in FIG. 4, the stimulation management unit 19 sets the duration of the generation period of the electrical pulse to be substantially the same as that of the non-determination period.

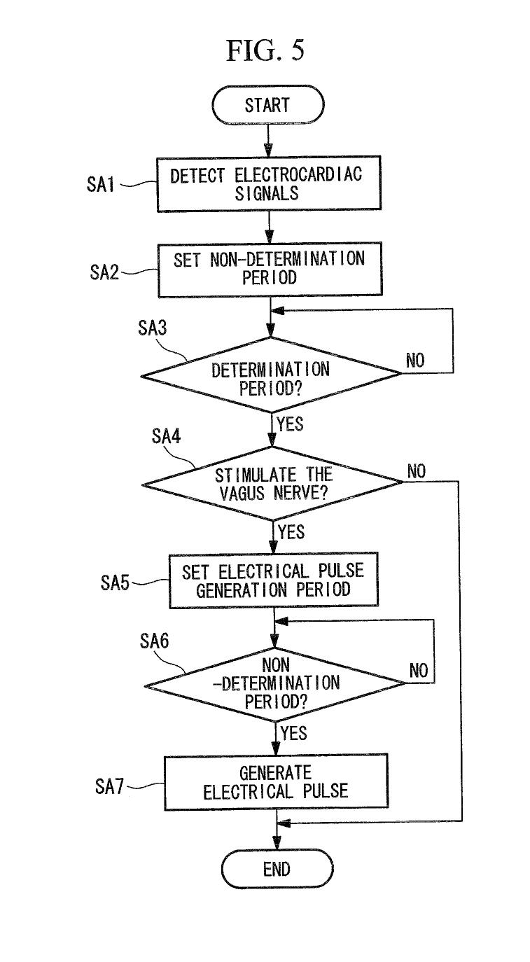

The operation and effects of the nerve stimulation apparatus 100 configured in this way will be described below with reference to a flowchart shown in FIG. 5.

According to the nerve stimulation apparatus 100 of this embodiment, the detector 11 is operated to continuously detect the electrocardiac signal from the respective electrodes 21 attached to the heart H via the respective cardiac leads 20 (Step SA1). The detector 11 detects the timing information of the beating of the heart H from the electrocardiac signal and outputs it to the controller 15.

In the controller 15, for example, a period from the point at which the electrocardiac signal containing the R wave is detected by the detector 11 to the point at which the electrocardiac signal containing the T wave is detected by the detector 11 is set as the non-determination period by the decision unit 17, and a period from the point immediately after the electrocardiac signal containing the T wave is detected to the point immediately before a detection signal containing the R wave appearing thereafter is detected is set as the determination period by the decision unit 17 (Step SA2). By doing so, it is possible to prevent the electric potential variations due to pacing, spontaneous cardiac activity, or the like from being undesirably determined.

In addition, the decision unit 17 determines whether the present time is in the determination period (Step SA3), and if it is in the non-determination period, the decision unit 17 waits for the determination period (Step SA3 “NO”). If it is in the determination period (Step SA3 “YES”), then the decision unit 17 determines the activity state of the heart H on the basis of the timing information of the beating of the heart H that is detected and sent by the detector 11 during the determination period (Step SA4).

If the decision unit 17 determines that the activity state of the heart H is in the normal state, the stimulation of the vagus nerve V is not performed (Step SA4 “NO”). Conversely, if the decision unit 17 determines that the activity state of the heart H is in the tachycardiac state, stimulation of the vagus nerve V is performed (Step SA4 “YES”).

If the vagus nerve V is to be stimulated, first, the stimulation management unit 19 sets the generation period of the electrical pulse in accordance with the activity state of the heart H that has been determined by the decision unit 17 (Step SA5).

In this case, the stimulation management unit 19 controls the generation period of the electrical pulse so as to be included within the non-determination period. For example, as shown in FIG. 3, the generation period of the electrical pulse is set to have a shorter period than the non-determination period. Thereby, since the generation of the electrical pulse is stopped after the non-determination period is ended and the determination period is started, it is possible to prevent the interference of the electrical pulse with the detection circuit in the detector 11 via the living body during the determination period.

Next, the stimulation management unit 19 determines whether the present time is in the non-determination period (Step SA6), and if it is in the determination period, the decision unit 17 waits for the non-determination period (Step SA6 “NO”). If it is in the non-determination period (Step SA6 “YES”), then the stimulation management unit 19 outputs to the nerve stimulation unit 13 the generation signal for the electrical pulse, the generation period of which has been controlled.

The nerve stimulation unit 13 generates the electrical pulse in accordance with the instruction from the stimulation management unit 19, and supplies the electrical pulse to the electrode 31 via the nerve stimulation lead 30 during the set generation period (Step SA7). Thereby, the vagus nerve V, to which the electrode 31 is attached, is electrically stimulated and the heart rate of the heart H is regulated to lower values.

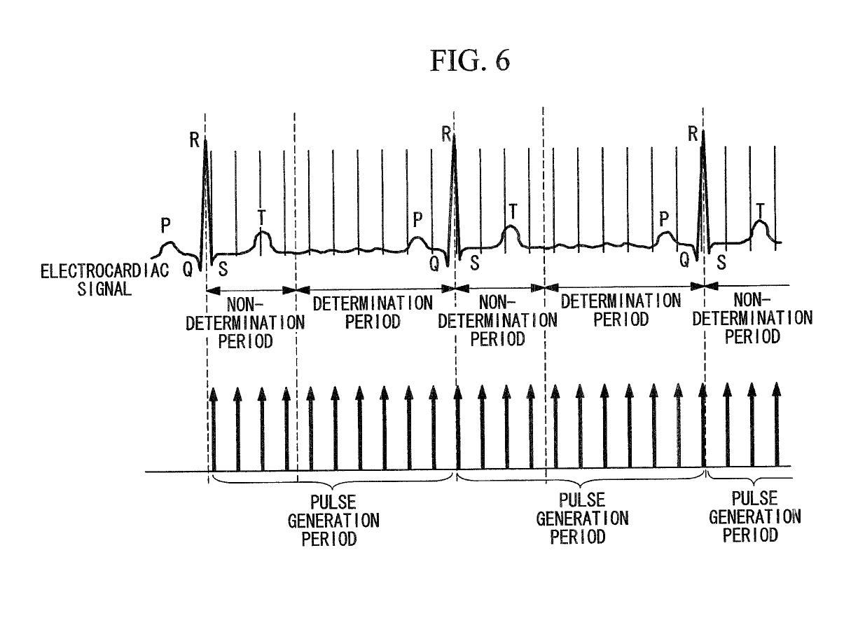

As a a reference example, for example, as shown in FIG. 6, even if the electrical pulse is generated during the determination period of the activity state of the heart H by the decision unit 17, the electrical pulse interferes with the detection circuit of the detector 11 via the living body during the determination period. In this case, the electrical pulse is included in the electrocardiac signal that is to be detected with the detector 11, which would end up being determined to be a change in the activity of the heart H by the decision unit 17.

According to the nerve stimulation apparatus 100 of this embodiment, since the generation period of the electrical pulse is controlled so as to be included within the non-determination period by the stimulation management unit 19 and the interference of the electrical pulse with the detection circuit of the detector 11 via the living body during the determination period is prevented, it is possible to avoid the inclusion of the electrical pulse into the electrocardiac signal that is detected with the detector 11 during the determination period. Therefore, it is possible to correctly determine, with the decision unit 17, the activity state of the heart H on the basis of the correct electrocardiac signal that has been detected by the detector 11 without the influence of the electrical pulse. Thereby, it is possible to stimulate the vagus nerve V at an appropriate timing.

In this embodiment, although the stimulation management unit 19 may set the duration of the non-determination period to be substantially the same as that of the generation period of the electrical pulse as long as the generation period of the electrical pulse is controlled so as to be included within the non-determination period, it is desirable that the generation period of the electrical pulse be set by also taking the position of the vagus nerve V to be stimulated into consideration.

In other words, depending on the position of the vagus nerve V to be stimulated, a certain time interval is needed for the electrical pulse, after it has been generated, to interfere with the detection circuit of the detector 11 via the living body. Thus, as shown in FIG. 4, when the durations of the non-determination period and the generation period of the electrical pulse are set to be substantially the same, if the position of the vagus nerve V to be stimulated is distant from the detector 11, the electrical pulse that is generated during the non-determination period may interfere with the detection circuit of the detector 11 during the following determination period.

In this case, the electrical pulse is included in the electrocardiac signal that is detected by the detector 11 during the determination period, which would end up being determined to be a change in the activity of the heart H by the decision unit 17. Thus, by considering the position of the vagus nerve to be stimulated, and by setting the duration of the generation period of the electrical pulse to be shorter than that of the non-determination period, it is possible to correctly determine the activity state of the heart H even when the interference of the electrical pulse is delayed.

This embodiment can be modified as follows.

In this embodiment, although the stimulation management unit 19 controls the generation period of the electrical pulse so as to be included within the non-determination period that has been preset by the decision unit 17, as a first modification for example, the decision unit 17 may extend the non-determination period, which has been set already, in accordance with the generation period of the electrical pulse that is required by the stimulation management unit 19 depending on the determination result of the activity state of the heart H.

In this case, the stimulation management unit 19 may set the necessary generation period of the electrical pulse and output it to the decision unit 17. In addition, when the generation period of the electrical pulse, which is sent from the stimulation management unit 19, exceeds the preset non determination period, the decision unit 17 may extend the non-determination period so as to include the generation period of the electrical pulse.

By doing so, if it is required to generate the electrical pulse outside the preset non-determination period in the stimulation management unit 19 as a result of the determination of the activity state of the heart H by the decision unit 17, by extending the non-determination period, it is possible to allow the generation period of the electrical pulse to be included within the extended non-determination period. Thereby, it is possible to stimulate the vagus nerve correctly and sufficiently while preventing the influence of interference of the electrical pulse, without reducing the necessary generation period of the electrical pulse.

As a second modification, the decision unit 17 may vary the non-determination period in accordance with the generation conditions of the electrical pulse. The generation conditions of the electrical pulse include, for example, the position of the vagus nerve V to be stimulated, the timing for generating the electrical pulse, and so forth.

These generation conditions of the electrical pulse vary depending on the treatment of the patient with cardiac disease, and the influence of the interference of the electrical pulse with the detection circuit of the detector 11 also vary depending on the treatment status. Therefore, by varying the non-determination period in accordance with the generation conditions of the electrical pulse by the decision unit 17, it is possible to perform nerve stimulation appropriate for the treatment status. In addition, even when the interference of the electrical pulse is delayed by changing the position of the vagus nerve V to be stimulated etc., by making the non-determination period sufficiently longer than the generation period of the electrical pulse in the decision unit 17, it is possible to correctly determine the activity state of the heart H by preventing the influence of interference of the electrical pulse.

Although the embodiment of the present invention has been above in detail with reference to the drawings, the specific configurations are not limited to these embodiments, and design alterations and the like within a range that does not depart from the sprit of the present invention are encompassed. For example, the above-mentioned embodiment has been described in terms of an example where three cardiac leads 20 are used. However, the number of cardiac leads 20 may be one or two, and these cardiac leads 20 may be electrically connected to desired positions on the heart H.

In addition, in the above-mentioned embodiment, although the determination period and the non-determination period are set by the decision unit 17, instead of this, the determination period and the non-determination period may be set by the stimulation management unit 19. In this case, the set determination period and the non-determination period may be input to the decision unit 17 by the stimulation management unit 19, and the activity state of the heart H may be determined by the decision unit 17 in accordance with the determination period and the non-determination period. Alternatively, the stimulation management unit 19 may control the determination timing of the activity state of the heart H by the decision unit 17 on the basis of the set determination period and the non-determination period.

What is claimed is:

1. A nerve stimulation apparatus comprising:
a detector that detects an electrocardiac signal;
a nerve stimulation unit that generates an electrical pulse for stimulating the vagus nerve; and
a controller that determines a cardiac activity state on the basis of the electrocardiac signal detected by the detector and controls the nerve stimulation unit on the basis of the determination result;
wherein the controller has a determination period for determining the cardiac activity state and a non-determination period for not determining the cardiac activity state, and wherein the controller allows the electrical pulse to be generated from the nerve stimulation unit while controlling a generation period of the electrical pulse so as to be included within the non-determination period.

2. A nerve stimulation apparatus according to claim 1, wherein the non-determination period is a period from the point at which the electrocardiac signal containing the R wave is detected by the detector to the point at which the electrocardiac signal containing the T wave is detected by the detector.

3. A nerve stimulation apparatus according to claim 1, wherein the controller extends the non-determination period according to the determination result of the cardiac activity state.

4. A nerve stimulation apparatus according to claim 1, wherein the controller varies the non-determination period according to a generation condition of the electrical pulse.
